(19) United States
(12) Patent Application Publication
(10) Pub. No.: US 2026/0174683 A1
(43) Pub. Date: Jun. 25, 2026

(54) CHEWABLE COMPOSITION COMPRISING A GUMMY MATRIX AND COMPRISING MELATONIN, SODIUM ALGINATE, AND CALCIUM LACTATE

(71) Applicant: S.I.I.T. S.R.L.-SERVIZIO INTERNAZIONALE IMBALLAGGI TERMOSALDANTI, Trezzano sul Naviglio (MI) (IT)

(72) Inventors: Andrea Domizio COSTA, Trezzano sul Naviglio (MI) (IT); Elena MADARO, Trezzano sul Naviglio (MI) (IT)

(21) Appl. No.: 19/125,647

(22) PCT Filed: Oct. 4, 2023
(86) PCT No.: PCT/IB2023/059975

§ 371 Date: Apr. 29, 2025

(30) Foreign Application Priority Data
Oct. 31, 2022 (IT) 102022000022413

(51) Int. Cl. A61K 9/00; A23G 3/36; A61K 9/16; A61K 31/185; A61K 31/4045; A61K 31/734

(52) U.S. Cl. CPC A61K 9/0056; A23G 3/364; A61K 9/167; A61K 31/185; A61K 31/4045; A61K 31/734

(57) ABSTRACT

It is an object of the present invention to provide a composition in a solid form, preferably in the form of a multilayer gummy with differentiated release of active ingredients.

### TECHNICAL BACKGROUND

The present invention relates to a multilayer chewable formulation with differentiated release of active ingredients. A chewable formulation is defined as a pharmaceutical form that is taken orally and comminuted and/or reduced into small pieces or fragments by the mechanical action of the teeth before being ingested.

The administration of drugs, and particularly of active ingredients in supplement form occurs, mainly, by the oral route as this route of administration appears to be the most convenient and favorable method.

In the field of oral administration, multilayer formulations for the differentiated release of one or more active ingredients in tablet form, preferably oral swallowable tablets, are known in the art. Oral swallowable tablets are tablets that can be swallowed or ingested. Oral swallowable tablets are prescribed and administered in their original shape and size, preferably using a liquid such as, for example, water to facilitate their intake.

Controlled release of the substances and/or compounds and/or active ingredients in a multilayer tablet is achieved through the design and formulation of the various layers of the tablet so that each one dissolves or disintegrates at different rates in the gastrointestinal tract, thus allowing the gradual release of the substances or active ingredients into the body.

The most widely used method of achieving controlled release in a multilayer tablet is to formulate one immediate-release layer and one or two prolonged-release layers. In this approach, the tablet consists precisely of two or more layers, one of which contains the immediate-release active ingredient, while the other layers are designed to release the active ingredient more slowly and gradually. The delayed-release layers contain excipients that delay the disintegration or dissolution of the intact swallowed tablet.

A multilayer tablet for controlled release of active ingredients is known; in such a tablet there are active ingredients with formulations such that they can be released at different times; in particular, the release time of the active ingredient can be immediate, fast, or delayed. Depending on the type of release, the active ingredients are dispersed in different layers of the tablet. However, tablet administration may be difficult for some types of users, particularly children and the elderly. In addition, in order to administer sufficient amounts of some active ingredients, it may be necessary to increase the size of the tablet, resulting in greater difficulty in oral intake. On the other hand, the excipients used in tablet technique have specific requirements that do not allow for much variation in existing formulations. To date, in common galenic practice, it is not possible to achieve controlled release in chewable or or soluble tablets or other pharmaceutical forms because differentiated release assumes that the tablet arrives intact in the gastrointestinal tract and then gradually releases the actives as a result of erosion of the tablet by gastrointestinal fluids. Disintegration of the tablet by the mechanical action of the teeth could in fact impair the controlled release of the actives carried in the tablet matrix and/or, even worse, result in a bitter or unpleasant taste because the actives present, once the tablet is disintegrated, come in contact with saliva, spread in the mouth or on the tongue resulting in an unpleasant taste.

In this context, the Applicant realized that known oral forms of administration should be improved to achieve greater flexibility in the formulation of the active ingredients, preferably they should be improved to make sure that even a chewable form of administration, such as a candy, preferably a gummy candy, which is swallowed only after being previously comminuted or reduced into small pieces by the mechanical action exerted by the teeth, can boast of a differentiated release, achieved by modifying the release of the active itself and not through the excipients, carried in the tablet matrix, which modulate its disintegration and dissolution.

In light of the above, therefore, the need persists for operators in the industry to solve the above problems in a simple and cost-effective way.

### Purposes of the Invention

The purpose of the present invention is to provide formulations of active ingredients for oral intake that allow even high dosages to be administered with a single daily administration, or with a minimum number of administrations, of active ingredients with differentiated release.

This composition should also be able to administer multiple active ingredients with controlled release.

These purposes are achieved by means of the present invention which relates to a chewable composition and a process of tablet production, according to the united claims.

Preferred embodiments are the subject of dependent claims.

In the context of the present invention, by active ingredients or formulations of active ingredients is meant to include, without limitation, active ingredients with or without pharmacological activity, food ingredients, natural extracts, for example of plant origin, or molecules and polymers of natural or synthetic origin.

In the context of the present invention composition(s) is meant to include, without limitation, pharmaceutical compositions, compositions for dietary supplements, compositions for medical devices Reg. EU 745/2017, foods for special medical purposes FSMPs, or food compositions.

According to one aspect, the invention relates to a chewable composition comprising a gummy matrix and at least one active ingredient in said gummy matrix, characterized by the fact that said gummy matrix presents at least two layers, each of said layers containing one or more of the same or different active ingredients, each layer being formulated with said at least one active ingredient to provide different forms of controlled release of said at least one active ingredient.

According to a further aspect, the invention relates to a process for the preparation of said composition comprising the preparation of at least two layers of different formulations of one or more active ingredients, the latter being the same or different from each other, in a single composition, i.e., gummy candy, preferably a chewable gummy candy.

A chewable candy (or bonbon, or sweetie, or dragée or pill, or lonzege) is suitable to be chewed and, therefore, swallowed only after it has been previously comminuted or reduced into small pieces by the mechanical action exerted by the teeth. The same is true for a chewable gummy candy where, the controlled or differentiated release, of the actives in it, is achieved by modifying the release of the actives themselves and not through the excipients, carried in the candy matrix which, instead, modulate the disintegration and dissolution of the candy.

Preferably, in the composition of the present invention, there may be only two layers superimposed on each other, particularly when there are active ingredients, for example, two active ingredients, either the same or different from each other, to be carried and these active ingredients do not exhibit interactions with each other, then there is a multilayer gummy with only two layers. In practice, if the active ingredients present in layers superimposed on each other do not give rise to chemical-physical interactions such as to degrade or deteriorate the composition of the multilayer gummy, then said superimposed layers can be in contact with each other and there is no need to introduce an additional separation layer between said layers.

Preferably, in the composition of the present invention, there may be three layers superimposed on each other.

According to one aspect, at least one of the layers acts as a separator between the remaining layers of the composition. This embodiment is particularly useful when interactions may occur between active ingredients present in two layers and when you have the same active ingredient formulated in two different release forms, for example, a retard granule in one layer and an immediate-release form in the other layer.

The separator layer may be free of active ingredients or may contain active ingredients that do not interact with those in adjacent layers.

A typical embodiment provides an immediate-release first layer, a slow-release second layer, and an intermediate-release third layer, the latter placed between said first and second layers.

As mentioned above, in one embodiment the same active ingredient is present in at least two layers in differentiated release form, and in other embodiments each layer contains different active ingredients.

The Applicant has, in addition, realized that it is possible to act on the release times of the actives by granulating, appropriately the actives whose dosage is to be modified, and by appropriately choosing the components of the gummy matrix. Since this is a chewable form of administration, which is therefore subject to the comminution of the matrix that composes it, it is essential to modify the release of the active ingredient itself (a condition that is not configured as mandatory and necessary, in a swallowable pharmaceutical form whose modified release is achieved by using appropriate excipients that are carried in the matrix itself of the swallowable tablet and therefore requires that it be swallowed whole and not comminuted) so that a differentiated release of the active ingredients can be achieved.

In an embodiment, the active ingredients are provided in their standard or modified form through granulation processes, and the formulation of the gummy base of at least two layers is different to provide differentiated release of the active ingredient(s) present in said layer. In an embodiment, the formulation of the gummy base of at least two layers is different to provide a differentiated release of the active ingredient(s) present in said layer.

The invention has many advantages.

Gummies are traditionally accepted by the public. Their chewability allows to operate with even high dosages of active ingredients, as they do not have to be swallowed whole.

Consider that a swallowable tablet can reach a maximum weight of 1.8 grams, while a “gummy” form of administration can reach 8 grams. The chewable composition is particularly suitable for patients with dysphagia and swallowing problems, both young and old. By varying the formulations of the gummy matrix, for the same formulation of active ingredients, it was surprisingly possible to obtain a release that varied, as far as the retard layer was concerned, from a minimum of 2 hours to a maximum of 6 hours.

For the preparation of the preferred compositions of the invention, the granulate of the invention may be mixed with excipients known to the person skilled in the art for making gummy candies, chewable tablets, capsules or chewing gum. By way of example, in the preparation of gummy candies can be used for example sugars, including glucose or fructose syrup, modified starches, gelatins, pectins, water, vegetable oils, and flavorings, coloring agents and sweeteners may also be used.

### BRIEF DESCRIPTION OF THE FIGURES

FIG. 1 shows the Vitamin C multilayer gummy.

FIG. 2, FIG. 3A and FIG. 3B show photographs of the outcome of the test to verify the gelling property of the granulate containing magnesium lactate.

FIG. 4 shows Melatonin:water=1:2 after 60 seconds.

FIG. 5 shows Melatonin:acidified aqueous solution (pH 3-3.5)=1:2 after 60 seconds.

### DESCRIPTION OF THE INVENTION

The Applicant, after intensive and prolonged research and development activity, has solved the problems discussed above and achieved the above-mentioned purposes by means of a composition comprising a multilayer matrix i.e., gummy base, i.e., comprising at least two layers, and one or more active ingredients according to the united claims.

Preferably, said composition comprises a multilayer matrix i.e., gummy base, i.e., comprises at least two layers, and one or more standard or granulated active ingredients so as to achieve a differentiated release of the same multiple active ingredients. The composition allows prolonged release of at least part of at least one active ingredient. The composition is advantageously in the form of gummy candy, particularly multilayer gummy candy with superimposed layers.

“Multilayer composition” here means a gummy candy formed by at least two layers, preferably superimposed on each other, comprising the different active ingredients; preferably, said gummy candy is formed by at least three layers. Preferably, the multilayer composition of the present invention may have any shape, for example, it may be cubic, parallelepiped, cylindrical, ellipsoidal or spherical in shape.

All components of the composition of the invention, including excipients and any coating film of the composition or candy are known in the art.

The components of the gummy matrix are known per se in the art.

The gelling compound is chosen from gelatin, pectin, food starch, carrageenan, gum, agar-agar or any other suitable gelling agent, or a combination thereof. For example, the gelling agent (i.e., the binder) may consist of or comprise gelatin-based products derived from animal sources, or from plant sources, and also any other suitable gelatin-based product. An example of a suitable animal gelatin product is PHARMA GRADE BOVINE GELATINE 150 BLOOM 30M from Lapi Gelatine. An example of a suitable vegetable gelatin product is Agar Agar, for example, the one available from Gino Biotech. Examples of pectin-based products, may include E440 pectin products made from fruit, or any other suitable pectin products. For example, CESAPECTIN® HMSS NA products from Tate & Lyle and GENU® pectin type VIS product, derived from citrus, from CPKelco are suitable. Examples of gelling compounds that include starchy ingredients are corn starch, rice or pea starch, potato starch, starch derivatives such as modified starches, preferably acetylated pea starch such as Roquette's CLEARGUM® LG 7015 product.

Examples of gelling compounds are potato starch, tapioca starch, and carrageenan. Among gelling compounds, hypromellose cannot be used because it is a stable polymer only at moderate temperatures (up to about 60° C.) and therefore degrades when subjected to the high temperatures applied during the baking process of a gummy candy. In fact, hypromellose is a hydrophilic gelling polymer used mainly in the pharmaceutical industry in the formulation of chewable or swallowable tablets as a water retention agent, stabilizer, thickener, or as a modulator of release of active ingredients, in processes that do not involve the application of temperatures >60° C. Therefore, hypromellose cannot be considered a suitable alternative to replace the gelling agents of choice used in a gummy candy (gelatin, starch, pectin). On the other hand, gelatin, because it requires to be dissolved in water or other liquid before it can be used as a gelling agent, cannot be used in solid and anhydrous pharmaceutical forms such as swallowable or chewable tablets, capsules, and solid powder mixtures in general. For these reasons, hypromellose and gelatin cannot be considered equivalent gelling compounds.

According to one embodiment, the composition is directed at sleep disorders and comprises melatonin, melatonin retard, and at least one additional active ingredient chosen from vitamin B6, valerian, lemon balm, hawthorn, passion flower, and mixtures thereof.

Preferably, melatonin with valerian and vitamin B6 are present in a “Fast” immediate-release layer, lemon balm and hawthorn in a “Normal” standard-release layer, and in the delayed-release, “Retard” layer is retard melatonin with passionflower. Preferably, melatonin has a titer of 98% or higher, more preferably 99% or higher.

Preferably, vitamin B6 (pyridoxine hydrochloride) is chosen, for example, with CAS No. 58 56-0 and molecular mass of about 205.64 g/mole.

Preferably, hawthorn is chosen from, for example, the species Crataegus pinnatifida, Crataegus monogyna Jacq. and/or Crataegus laevigate (Poir.) DC, preferably in its leaf and flower components, as an extract (e.g., extraction with an extraction solvent comprising 80% ethanol and 20% water) preferably dry extract. For example, 100 mg of extract is equivalent to about 400 mg of dry extract of hawthorn leaves and flowers. For example, a dry extract of hawthorn leaves and flowers contains about 1%-5%, e.g., at least 1.5% flavonoids (HPLC). Preferably, hawthorn is, for example, of the species Crataegus pinnatifida, is in the form of a dry extract titrated in vitexin (vitexin-2″-O-rhamnoside content), and has a titer from 1% to 4%, preferably from 2% to 3%, by weight (HPLC).

Preferably, valerian is for example from the species Valeriana Officinalis, in dry extract form, preferably hydroalcoholic dry extract (CAS No. 8057-49-6). Preferably, the extract is a dry extract, titrated in valerenic acids, e.g., valerenic acid, hydroxyvalerenic acid, and acetoxyvalerenic acid measured by HPLC, and has a titer from 0.5% to 2.5%, preferably from 0.8% to 1.5%, by weight (HPLC).

For example, extract is obtained from rhizome and roots with a water and ethanol solvent and a drug:extract ratio of about 3-4:1. The dry extract, such as powder or granules, has an average particle size of about 80-100 mesh (80 mesh=180 micrometers) and an average bulk density of about 0.70 g/ml.

Preferably, passion flower is for example of the species Passiflora Incarnata in dry extract form, preferably hydroalcoholic dry extract (CAS No. 729668-47-9).

Preferably, the extract is a dry extract, titrated in isovytoxin, e.g., isovitexin, vitexin, iso-orientin, and orientin, and has a titer from 1.5% to 6%, preferably from 3% to 4%, by weight (HPLC).

For example, extract is obtained from flowers with a water and ethanol solvent and a drug:extract ratio of about 4:1.

The dry extract, such as powder or granules, has an average particle size of about 80-100 mesh (80 mesh=180 micrometers) and an average bulk density (bulk density) of about 0.78 g/ml.

Preferably, lemon balm is for example of the species Melissa Officinalis in a dry extract form, preferably hydroalcoholic dry extract (CAS No. 84082-61-1). Preferably, the extract is a dry extract, titrated in hydroxycinnamic derivatives, e.g., expressed as rosmarinic acid, and has a titer from 5% to 25%, preferably from 10% to 15%, by weight (HPLC).

For example, extract is obtained from leaves with a water and ethanol solvent and a drug:extract ratio of about 4:1. The dry extract, such as powder or granules, has an average particle size of about 75-300 mesh (75 mesh=200 micrometers).

In an embodiment, the mass ratio (w/w) (calculated on the whole multilayer composition) of melatonin/vitamin B6/valerian/hawthorn/passion flower/lemon balm may preferably be about 1/6.2/100/176/100/50.

The chewable compositions of the invention may contain from 0.5 to 10 mg of melatonin, preferably from 1 to 8 mg of melatonin, e.g., 1 mg, 2 mg, 3 mg, 4 mg, or 5 mg.

According to a preferred embodiment, the gummy candy provides three layers, each layer having a different formulation. In one embodiment two layers contain melatonin, one layer being immediate-dissolving and one being slow-dissolving or delayed-dissolving, preferably the immediate-dissolving layer comprises melatonin, vitamin B6, and valerian, while the slow-dissolving layer comprises melatonin in retard granule form, preferably 10-15%, and passion flower.

A method for granulating melatonin together with calcium lactate and sodium alginate is reported in the experimental section. The granule obtained is present in at least one layer of the composition or gummy candy of the present invention.

Among all soluble alginate salts, sodium alginate was combined with a soluble salt of a bivalent cation. Among all possible bivalent cations, the Applicant selected Ca2+, both according to affinity and taking into account safety. Of all the soluble salts of a bivalent cation, the choice was made in favor of calcium lactate because it was the result of careful selection reasoning that took into account the physicochemical properties of calcium lactate and the other components present in the composition of the granulate (sodium alginate and melatonin). Among all possible calcium salts, calcium lactate was chosen because of a number of characteristics that make calcium lactate the salt of choice for the preparation of melatonin granulate, which is the object of the present invention: i) good solubility in water; ii) pH of the 5% solution of calcium lactate in water ranging from 6 to 8; iii) Ca2+ titer ranging from 12% to 18%, preferably ranging from 13.4% to 14.5%.

All components of the granulate of the invention are known in the art and commercially available. Preferably, the melatonin has a titer equal to or greater than 95%, more preferably equal to or greater than 98%, even more preferably equal to or greater than 99%, e.g., 99.5%. Preferably, the granulate of the invention include melatonin from 1% to 20% by weight of melatonin with respect to the total weight of the granulate, preferably from 1.5% to 15%, more preferably from 2% to 10%, e.g., 2.5%; 3%; 3.5%; 4%; 4.5% or 5%.

Preferably, the granulate present in at least one layer of the composition or gummy candy of the present invention comprises sodium alginate from 20% to 60%, preferably from 40% to 55%, more preferably from 45% to 50%, e.g., about 46%; 47%; 48%; or 49% of sodium alginate with respect to the total weight of the granulate.

Preferably, the granulate present in at least one layer of the composition or gummy candy of the present invention comprises from 30% to 60% of calcium lactate, preferably from 40% to 55%, more preferably from 45% to 50%, e.g., about 46%; 47%; 48%; 49% of calcium lactate.

Preferably, the granulate present in at least one layer of the composition or gummy candy of the present invention comprises:
from 1% to 20% by weight of melatonin, with respect to the total weight of the granulate, preferably from 1.5% to 15%, more preferably from 2% to 10%;
from 20% to 60% by weight of sodium alginate, with respect to the total weight of the granulate, preferably from 40% to 55%, more preferably from 45% to 50%, e.g., about 49%; and
from 30% to 60% by weight of calcium lactate, with respect to the total weight of the granulate, preferably from 40% to 55%, more preferably from 45% to 55%, e.g., about from 49% to 50%.

Preferably, the preparation of the granulate present in at least one layer of the composition or gummy candy of the present invention comprises or, alternatively, consists of the following steps:
a. a wet granulation of a mixture of (i) melatonin, in an amount by weight from 1% to 20%, e.g., about 2%, or 10% by weight, with respect to the total weight of the granulate, (ii) sodium alginate, in an amount by weight from 20% to 60%, e.g., about 45%, or 49% by weight, with respect to the total weight of the granulate, and (iii) calcium lactate, in an amount by weight from 30% to 60%, e.g. 45%, or 49% by weight, with respect to the total weight of the granuleate, to form granules intended to be coated to give coated granules;
b. a coating of the granules obtained from step (a) through two sequential steps to give coated granules:
b′ a granulation of the granules from passage (a) with calcium lactate; and
b″ a granulation of the granules from the passage (b′) with sodium alginate.

In step (a), a wet granulation is made by mixing from 1% to 20% by weight of melatonin with from 20% to 60% by weight of sodium alginate, and with from 30 to 60% by weight of calcium lactate and water, according to known techniques.

The granules obtained in step (a) are dried, e.g., in an oven, preferably at a temperature from 40° C. to 80° C., more preferably from 50° C. to 70° C., e.g., at 60° C., and for a time preferably from 2 hours to 12 hours, more preferably from 4 hours 10 hours, e.g., 8 hours. Preferably, the dried granules undergo a calibration step using, for example, a mesh or sieve with a mesh size from 0.5 mm to 2.5 mm, preferably from 1 mm to 2 mm, e.g., 1, 2 mm, or 1.4 mm, or 1.6 mm.

Said dried granules from step (a) are, then, coated by step (b) through the two subsequent granulations (b′) and (b″); first the granules are treated with calcium lactate in step (b′) to obtain calcium lactate-coated granules, then the latter are further coated by sodium alginate in step (b″). The granules obtained after granulation in each of steps (a), (b′) and (b″) are dried according to known techniques.

Preferably, the granulations in steps (b) are fluidized bed or slurry granulations.

Alternatively, the process for preparing the granuleate can be accomplished by a single granulation of a mixture of melatonin, sodium alginate and calcium lactate, followed by a single coating step.

Preferably, the preparation of the granulate present in at least one layer of the composition or gummy candy of the present invention comprises or, alternatively, consists of the following steps:
a. a wet granulation of a mixture of (i) melatonin, in an amount by weight from 1% to 20%, e.g., about 2%, or 10% by weight, with respect to the total weight of the granulate, (ii) sodium alginate, in an amount by weight from 20% to 60%, e.g., about 45%, or 49% by weight, with respect to the total weight of the granuleate, and (iii) calcium lactate, in an amount by weight from 30% to 60%, e.g. 45%, or 49% by weight, with respect to the total weight of the granulate, to form granules intended to be coated to give coated granules;
b. a coating of the granules obtained from step (a) through one coating step with a mixture of sodium alginate and calcium lactate, to give coated granules.

Preferably, the granules have a size ranging from 0.8 mm to 1.5 mm, preferably from 1 mm to 1.4 mm.

Some details of the process are provided in the Experimental Section below, for illustrative purposes only.

The granulate described herein allows for a prolonged time release of melatonin after it is taken by a subject in need of melatonin. Said granulate can be used as such, e.g., it can be included in gelatin capsules to be administered for the treatment of sleep disorders and for the reduction of the time required to fall asleep.

Preferably, the granulate described herein can be used for the preparation of pharmaceutical compositions and dietary supplements with controlled-release of melatonin.

According to another of its aspects, it is also an object of the invention an oral pharmaceutical composition and/or dietary supplement comprising the granulate of the invention together with one or more conventional excipients and/or vehicles.

According to a preferred embodiment, the compositions of the invention are in the form of gummy candies, chewable tablets, capsules or chewing gum.

For the preparation of the preferred compositions of the invention, the granulate of the invention may be mixed with excipients known to the person skilled in the art for making gummy candies, chewable tablets, capsules or chewing gum. By way of example, sugars, including glucose syrup, modified starches, gelatins, pectins, water, vegetable oils may be used for the preparation of gummy candies, for example, and flavoring, coloring and conventional coating agents may also be added.

Alternatively, the compositions of the invention may be in the form of tablets, hard capsules, soft capsules, granules, fine granules, powders, tablets, syrups, emulsions, suspensions and solutions suitable for oral administration. Other suitable pharmaceutical forms may also be used.

Such compositions can be taken alone, for example, when in the form of gummy candies, chewable tablets or chewing gum, or with water when in capsule form, or, especially when in the form of powders or granules, they can be mixed with other foods, for example, with yogurt, creams, gels and the like.

Alternatively, the compositions of the invention can be in ready-to-use beverage form, such as in the form of drinking sachets, of the “stick pack” type, in this case preferably in cream or gel form.

In addition to those already mentioned, the types of pharmaceutical and food additives used in preparing the compositions of the invention, the content ratios of additives to active ingredients, and the methods of preparing the pharmaceutical composition or food supplement may be appropriately selected by the person skilled in the art.

For conventional vehicles and excipients, organic or inorganic substances, or solid or liquid substances may be used as excipients and vehicles, provided they are edible, physiologically acceptable, and compatible with all other components of the composition.

As mentioned, the person skilled in the art is perfectly capable of selecting the most suitable vehicles and excipients for the preparation of the composition.

As examples, organic or inorganic substances, or solid or liquid substances may be used as excipients and vehicles, provided they are edible and pharmaceutically acceptable. Examples of excipients used in the preparation of solid pharmaceutical compositions include, for example, lactose, sucrose, starch, talc, cellulose, dextrins, kaolin, calcium carbonate, stearic acid or magnesium stearate, lactose, polyethylene glycol, mannitol, sorbitol, chelating agents, anti-caking agents, sweetening agents, preservative agents, and flavoring agents.

For the preparation of liquid compositions for oral administration, a conventional inert diluent such as water or an oil, such as a vegetable oil, may be used. The liquid composition may contain, in addition to the inert diluent, auxiliaries such as wetting agents, suspending agents, sweeteners, flavorings, colorants, and preservatives. The liquid composition may be included in capsules of an absorbable material, such as gelatin.

Sweeteners may be one or more natural sugars, optionally reduced, such as sucrose, dextrose, xylitol, mannitol, or sorbitol, or a synthetic product such as sodium saccharin, aspartame, acesulfame k, or sucralose. Acidifying agents may also be added.

Flavoring agents are pharmaceutically acceptable flavors and tastes of synthetic oils or natural oils, the latter extracted from plants, flowers, fruits, and combinations thereof, such as cinnamon, mint, anise, and citrus leaves, bitter almonds, citrus fruits, especially orange and/or lemon, tilia, vanilla, chocolate, and grapefruit oils. Chocolate, vanilla or eucalyptus flavorings and fruit essences, especially apple, pear, peach, strawberry, apricot, orange, lemon and grape, can also be used advantageously.

Examples of the composition of the invention are provided in the Experimental Section below, for illustrative purposes only.

Compositions of the invention may contain from 0.5 mg to 10 mg of melatonin, preferably 1 mg to 3 mg of melatonin, e.g., 1 mg or 2 mg.

As will be shown in the Experimental Section, the compositions of the invention comprising the granulate, particularly the compositions in the form of gummy candies, showed a dissolution profile particularly suitable for the treatment of sleep disorders, for the reduction of the time required to fall asleep and for the maintenance of the state of falling asleep.

The expression “sleep disorders” is intended here to include any sleep-related pathology, such as difficulty falling asleep, duration, consolidation, or quality of sleep, and preferably the expression identifies primary insomnia.

The granulate or compositions of the invention are nontoxic and are intended for administration to mammals, preferably humans, in any age group: from adolescents to the elderly.

The granulate or compositions of the invention should be administered shortly before bedtime, e.g., from 15 to 60 minutes earlier, preferably from 30 to 40 minutes earlier. Usually a dose from 1 mg to 3 mg of melatonin, e.g., 1 mg or 2 mg is sufficient to achieve the desired effect. However, these dosages may vary depending on the age, sex, and health status of the person being treated as well as the severity of the disorder.

According to another of its aspects, it is an object of the invention the use of the granulate and composition of the invention in the treatment of sleep disorders as defined above.

According to another of its aspects, it is an object of the invention a method for the treatment of sleep disorders as defined above, which comprises administering an effective amount of the granulate or composition of the invention to a subject in need thereof.

According to another of its aspects, it is also an object of the invention an association consisting of melatonin, sodium alginate and calcium lactate.

According to another of its aspects, it is also an object of the invention an association consisting of melatonin, sodium alginate and calcium lactate for use in a method for the treatment of sleep disorders, as defined above.

According to another of its aspects, it is also an object of the invention an association consisting of melatonin, sodium alginate and calcium lactate as the active ingredient of preferably prolonged-release pharmaceutical compositions for use in a method for the treatment of sleep disorders, as defined above.

According to another of its aspects, it is also an object of the invention an association consisting of melatonin, sodium alginate and calcium lactate, as the active ingredient of prolonged-release granulates, for use in a method for the treatment of sleep disorders, as defined above.

The invention will now be described in more detail in the Experimental Section that follows, for illustrative purposes and in no way limiting.

In one embodiment, the active ingredient is vitamin C and is present in one layer as a delayed-release granulated formulation and in another layer as readily available vitamin C powder.

In one embodiment, the composition is in the form of a three-layer candy, where in one Fast layer there are at least two compounds chosen from calcium pantothenate, vitamin pp niacinamide, vitamin B1 nitrate rocoat 33%, vitamin B2 rocoat 33% ribofl., vitamin B6 hydrochloride rocoat 33%, folic acid, vitamin B12 0.1% ws 429090, magnesium oxide cd.

A second Normal layer contains at least two compounds chosen from zinc bis-glycinate id, copper bis-glycinate id, iron fumarate coated, sodium selenite 45%, chromium chloride 5%, manganese sulfate H2O, potassium iodide, sodium molybdate 10% id.

A third Retard layer contains at least two compounds chosen from vitamin C granulate, vitamin A acetate 500, vitamin D3 100 cws, vitamin E acetate 50% type sd, biotin (vitamin H).

According to one embodiment, the composition comprises Iron and Vitamin C; in one layer Iron is present, preferably as Iron pyrophosphate microencapsulated with soy or sunflower lecithin, and maltodextrins; in a second Retard layer Vitamin C granulate is present. The two layers are separated by an additional buffer layer free of active ingredients.

In one embodiment the composition comprises Magnesium. In such embodiment a first Fast layer contains Magnesium oxide and maltodextrins, and in a second Retard layer there is anhydrous Magnesium Citrate.

In embodiments, the layers of the composition or candy may contain different dyes to highlight the different composition.

Sweeteners may be one or more natural sugars, optionally reduced, such as sucrose, dextrose, xylitol, mannitol, or sorbitol, or a synthetic product such as sodium saccharin, aspartame, acesulfame k, or sucralose. Acidifying agents may also be added.

As previously mentioned, the formulation of the gummy base or matrix affects the rate of release of the active ingredient. For example, the Applicant has verified that the addition of starch to the matrix formulation, ceteris paribus, slows the release kinetics, particularly for retard layers. The maximum amount of starch to be added can be up to 8%, preferably up to 5%; the minimum amount is the amount effective to slow the release kinetics. Example 11 shows values for a melatonin composition.

Preferred embodiments FRn are given below.
FR1. A chewable composition comprising a gummy matrix and at least one active ingredient in said gummy matrix, characterized in that said matrix has in at least two layers, each of said layers containing one or more active ingredients, each layer being formulated with said at least one active ingredient to provide different forms of controlled release of said at least one active ingredient.
FR2. The composition according to FR1, wherein there are three superimposed layers.
FR3. The composition according to FR1 or FR2, wherein at least one of the layers acts as a separator between the remaining layers.
FR4. The composition according to FR3, wherein the separator layer is free of active ingredients.
FR5. The composition according to any one of FR1-FR4, comprising a first immediate-release layer; a second slow-release layer and a third intermediate-release layer.
FR6. The composition according to any one of FR1-FR5, wherein the same active ingredient is present in at least two layers in a differentiated release form.
FR7. The composition according to any one of FR1-FR6, wherein each layer contains different active ingredients.
FR8. The composition according to any one of FR1-FR7, wherein the formulation of the gummy base of at least two layers is different to provide differentiated release of the active ingredient(s) in said layer.
FR9. A process for preparing the composition according to any one of FR1-FR8, which comprises the preparation of at least two layers of different formulations of one or more active ingredients in a single composition i.e. a gummy candy.
FR10. The process according to FR9, wherein different layers have different formulation of the said gummy matrix to provide differentiated release of said one or more active ingredients.

Preferred embodiments PEn are given below.
PE1. A chewable composition comprising a gummy matrix and at least one active ingredient in said gummy matrix, wherein said active ingredient comprises or alternatively consists of melatonin, sodium alginate, and calcium lactate.
PE2. The composition according to PE1, wherein said matrix comprises a slow-release granulate that comprises or alternatively consists of melatonin, sodium alginate, and calcium lactate.
PE3. The composition according to PE2, wherein said granulate comprises from 1% to 20% by weight of melatonin with respect to the total weight of the granulate, preferably from 1.5% to 15%, more preferably from 2% to 10%; from 20% to 60% by weight of sodium alginate with respect to the total weight of the granulate, preferably from 40% to 55%, more preferably from 45% to 50%; and; from 30 to 60% by weight of calcium lactate with respect to the total weight of the granulate, preferably from 40% to 55%, more preferably from 45% to 50%.
PE4. The composition according to any one of PE1-PE3, wherein said matrix has at least two layers, each of said layers containing one or more active ingredients, each layer being formulated with said at least one active ingredient comprising or, alternatively, consisting of melatonin, sodium alginate, and calcium lactate to provide different forms of controlled release of said at least one active ingredient.
PE5. The composition according to any one of PE1-PE4, wherein there are three superimposed layers in said matrix; preferably, at least one of the layers acts as a separator between the remaining layers; even more preferably, said separator layer is free of active ingredients.
PE6. The composition according to PE5, wherein said matrix comprises a first immediate-release layer, a second slow-release layer, and a third intermediate-release layer.
PE7. The composition according to any one of PE1-PE6, wherein the same active ingredient is present in at least two layers in a differentiated release form.
PE8. The composition according to any one of PE1-PE6, wherein each layer contains different active ingredients.
PE9. The composition according to any one of PE1-PE8, wherein the formulation of the gummy base of at least two layers is different to provide differentiated release of the active ingredient(s) in said layer.
PE10. A process for preparing the composition according to any one of PE1-PE9, which comprises the preparation of at least two layers of different formulations of one or more active ingredients in a single composition i.e. a gummy candy.
PE11. The process according to PE10, wherein different layers have different formulation of said gummy matrix to provide differentiated release of said one or more active ingredients.
PE12. The composition according to any one of PE1-PE9, wherein said composition is for use in a method of treatment of sleep disorders, preferably primary insomnia, for the reduction of the time required to fall asleep and for the maintenance of the state of falling asleep.

The invention will now be described in more detail in the Experimental Section below, for illustrative purposes and in no way limiting.

### Experimental Section

### Example 1

### Melatonin Granulation Process

### Step (a)

TABLE 1
Granulation in Diosna mod. P1/6 lab

| Ingredient | % by weight |
|---|---|
| Melatonin 99% titer | 2.0% |
| Sodium alginate | 49% |
| Calcium lactate | 49% |

Load in order sodium alginate, e.g., about 49% by weight, melatonin, e.g., about 2% by weight, and calcium lactate pentahydrate, e.g., about 49% by weight. Set impeller to 200 rpm and pour in flush purified water qs. After addition is finished, set chopper to 1000 rpm and impeller to 200 rpm for 5 min. Pass wet granulate through 3 mm mesh.

### Drying Phase

Dry the granules in an oven at 60° C. for 8 hours. LOD control=6.3%

### Calibration Phase

Calibrate through 1.2 mm mesh

### Step (b)

TABLE 2

| Ingredient | % by weight |
|---|---|
| Granules from step (a) | 98.12% |
| Sodium alginate | 0.44% |
| Calcium lactate | 1.44% |

Step (b′)

Granulation in Glatt Fluid Bed mod.Uniglatt

### Coating Solution Preparation

Solubilize calcium lactate in purified water q.s.

### Coating Phase

Load the granule obtained from step 1 into the fluidized bed basket. Spray the coating solution by setting the following parameters:

$$Inlet air Temp . = 40 ° C . - 45 ° C .$$

$$Product Temp . = 27 ° C . - 30 ° C .$$

$$Spray pressure = 1.5 bar$$

$$Liquid flow rate = 15 - 20 g / min$$

$$Air flow = Flap pos . 15$$

After finishing the coating solution, dry by setting the following parameters:

$$Inlet air Temp . = 60 ° C . - 65 ° C .$$

$$Product Temp . = 50 ° C . - 55 ° C .$$

$$Air flow = Flap pos . 15$$

$$LOD control = 6.5 - 7.5 \%$$

Step (b″)

Granulation in Glatt Fluid Bed mod.Uniglatt

### Coating Solution Preparation

Solubilize Sodium Alginate in Water

### Coating Phase

Load the granules obtained from step (b′) into the fluidized bed basket. Spray the coating solution by setting following parameters:

$$Inlet air Temp . = 40 ° C . - 45 ° C .$$

$$Product Temp . = 27 ° C . - 30 ° C .$$

$$Spray pressure = 1.5 bar$$

$$Liquid flow rate = 15 - 20 g / min$$

$$Air flow = Flap pos . 15$$

### After Finishing the Coating Solution Drying by Setting Following Parameters:

$$Inlet air Temp . = 60 ° C . - 65 ° C .$$

$$Product Temp . = 50 ° C . - 55 ° C .$$

$$Air flow = Flap pos . 15$$

$$LOD control = 6.5 - 7.5 \%$$

### Calibration Phase

Calibrate the granules through 1.2 mm mesh.

### Example 2

### Granulation Process

### Step (a)

TABLE 3
Granulation in Glatt Fluid Bed mod. Uniglatt

| Ingredient | % by weight |
|---|---|
| Melatonin 99% titer | 10.0% |
| Sodium alginate | 45% |
| Calcium lactate | 45% |

In this case, a single granulation step is made in Glatt Fluid Bed with all ingredients and 40% purified water.

### Coating Phase

Load melatonin, e.g., about 10% by weight, sodium alginate, e.g., 45% by weight, and calcium lactate, e.g., 45% by weight, into the fluid bed basket. Spray the purified water by setting the following parameters:

$$Inlet air Temp . = 40 ° C . - 45 ° C .$$

$$Product Temp . = 27 ° C . - 30 ° C .$$

$$Spray pressure = 1.5 bar$$

$$Liquid flow rate = 15 - 20 g / min$$

$$Air flow = Flap pos . 15$$

After Finishing the Wetting Phase with Purified Water, Dry by Setting Following Parameters:

$$Inlet air Temp . = 60 ° C . - 65 ° C .$$

$$Product Temp . = 50 ° C . - 55 ° C .$$

$$Air flow = Flap pos . 15$$

$$LOD control = 6.5 - 7.5 \%$$

### Calibration Phase

Calibrate the granulate through 1.2 mm mesh

The resulting granulate has the following specifications:

### Granulometry:

No more than 10%: ≥710 μm
80% between 125 μm and 710 μm
Not more than 10%: <125 μm
LOD (70° C.): 6.0%-8.0%.
Density: 0.4-0.6 g/ml
Flowability (100 grams): 5 25 sec.

### Example 3

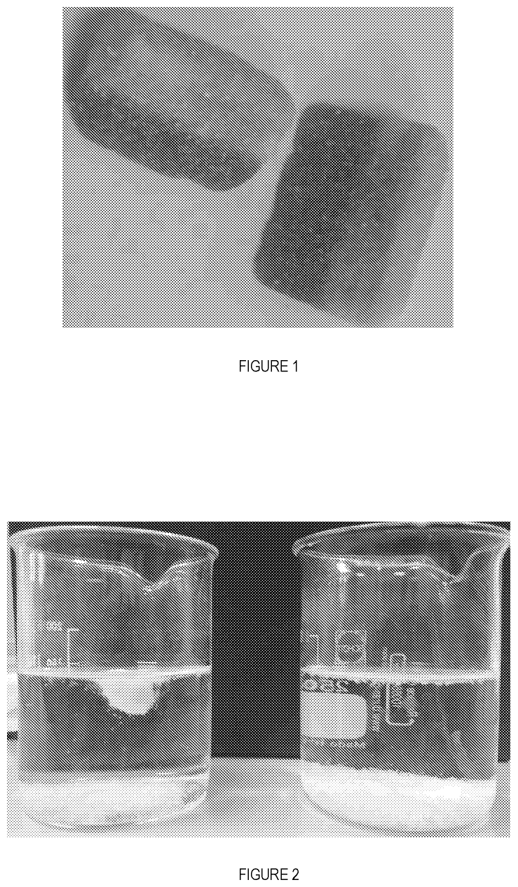

A retard layer of a multilayer gummy is prepared (FIG. 1).

Mould-form “menthol boluses”

Weight single candy Single candy: about 2 g

### Ingredients:

Granulate obtained from Example 1, glucose syrup, sugar, modified starches, water, vegetable oil (coconut, canola), flavoring, coloring agent: E133, coating agent: carnauba wax.

### Example 4

### Dissolution Tests

### Example 4.1

Dissolution test according to USP (500 mL 0.1 N HCl, 37° C., 100 rpm)

TABLE 4

| | | |
|---|---|---|
| | Candies from Example 3 | Candies from Example 3 |
| Time | divided into 2 parts | divided into small pieces |

| | | | |
|---|---|---|---|
| 30 | min | 19.3% | 29.6% |
| 2 | hours | 40.0% | 716% |
| 4 | hours | 54.7% | 76.2% |

### Example 4.2

Dissolution test (900 mL buffer pH 7, 37° C., 100 rpm)

TABLE 5

| | Candies from example 3 | Candies from Example 3 |
|---|---|---|
| Time | divided into 2 parts | divided into small pieces |

| | | | |
|---|---|---|---|
| 30 | min | 29.8% | 41.3% |
| 2 | hours | 57.0% | 55.6% |
| 4 | hours | 63.2% | 71.1% |

### Experimental Part

Alginates are naturally occurring linear copolymers extracted from the cell wall of the brown algae Laminaria and Ascophillum. Alginates from algae must undergo a series of treatments before they can actually be used for applications where their high purification is required. Since the alginate salts found in algae are insoluble, their transformation into sodium alginate, which is soluble instead, is necessary.

A relative alginate affinity scale was established for several bivalent cations of interest:

$$Pb 2 + > Cu 2 + Cd 2 + > Ba 2 + > Sr 2 + > Ca 2 + > Co 2 += Ni 2 += Zn 2 + > Mn 2 + > Mg 2 +$$

Among all the possible bivalent cations, Ca2+ was selected, both according to affinity and taking safety into account.

When using alginate gels, one of the main problems is low chemical stability.

Substances with a high affinity for them (such as, for example, phosphates or citrates) can sequester calcium ions and destabilize the initial gel. The gel may also be destabilized by the presence of other non-gelling cations.

For these reasons, the choice of calcium lactate does not seem obvious but is rather the result of careful selection reasoning that takes into account the chemical and physical properties of calcium lactate itself and the other components present in the granulate composition (sodium alginate and melatonin).

Of all the possible calcium salts, calcium lactate was chosen because of a number of characteristics that make calcium lactate the salt of choice for the preparation of melatonin granulate:
good solubility in water;
pH of 5% calcium lactate solution in water: 6-8;
Ca titer: from 13.4% to 14.5%.

The other possible calcium salts have been excluded either because they are insoluble in water (e.g., calcium carbonate) or because they are poorly soluble (e.g., calcium citrate) or because, although soluble, once dissolved in water they result in a pH value that is incompatible with the other two ingredients in the granulate composition (melatonin and sodium alginate).

The choice could also have fallen on a salt formed by the binding of lactic acid with a bivalent cation that is equally safe as Ca2+, namely Mg2+. The salt formed when lactic acid binds the bivalent cation Mg2+ is magnesium lactate. Magnesium lactate turns out to be very similar to calcium lactate in terms of its chemical and physical characteristics:
good solubility in water;
pH of 5% magnesium lactate solution in water: 6.5-8.5;
Mg titer: from 10.0% to 10.5%.

However, magnesium lactate, despite having similar chemical and physical characteristics to calcium lactate, is not considered to be a viable candidate, equivalent to calcium lactate in terms of functionality within the retard granuleate, which includes the presence of melatonin and sodium alginate, because the Mg2+ ion does not possess gelling properties, as the Ca2+ ion has.

To provide experimental evidence, a granulate was produced wherein calcium lactate was substituted 1:1 for magnesium lactate. The composition tested, the granulation process applied and the outcome of the test are given below:

| Ingredient | % |
|---|---|
| Melatonin | 10.00 |
| Sodium alginate | 45.00 |
| Magnesium lactate | 45.00 |

A single granulation step is made in Glatt Fluid Bed with all ingredients and 40% purified water.

### Coating Phase

Load into the fluid bed basket melatonin, e.g. about 10% by weight, sodium alginate, e.g. 45% by weight, magnesium lactate, e.g. 45% by weight.

Spray the purified water by setting the following parameters:

$$Inlet air Temp . = 40 ° C . - 45 ° C .$$

$$Product Temp . = 27 ° C . - 30 ° C .$$

$$Spray pressure = 1.5 bar$$

$$Liquid flow rate = 15 - 20 g / min$$

$$Air flow = Flap pos . 15$$

After the wetting phase with purified water is finished, dry by setting the following parameters:

$$Inlet air Temp . = 60 ° C . - 65 ° C .$$

$$Product Temp . = 50 ° C . - 55 ° C .$$

$$Air flow = Flap pos . 15$$

$$LOD control = 6.5 - 7.5 \%$$

### Calibration Phase

Calibrate the granulate through 1.2 mm mesh

The resulting granulate has the following specifications:

### Granulometry:

No more than 10%: ≥710 μm
80% between 125 μm and 710 μm
10 No more than 10%: <125 μm
LOD (70° C.): 6.0%-8.0%
Density: 0.45-0.65 g/ml
Flowability (100 grams): 5 25 sec.

The granulate obtained using magnesium lactate has chemical and physical characteristics superimposed on the granulate obtained using calcium lactate.

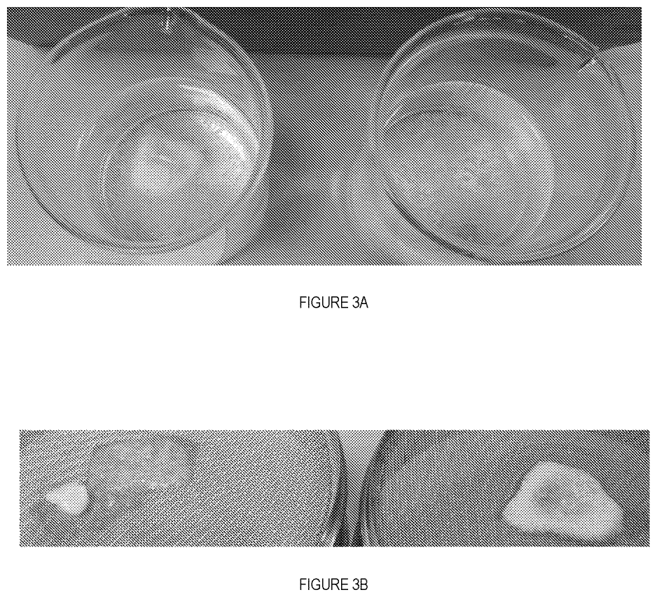

To test the gelling property of the granules containing magnesium lactate, which is responsible for the functionality of the melatonin granulate, 1 gram of granulate with calcium lactate and 1 gram of granulate with magnesium lactate were poured into 150 ml of HCl 0.1N to simulate the gastric environment (FIG. 2, FIG. 3A and FIG. 3B).

FIG. 2 (on the left granulate with magnesium lactate and on the right granulate with calcium lactate) shows the: behavior of the 2 granulate just poured into HCl 0.1N.

FIGS. 3A and 3B shows the granulate with Mg lactate and the granulate with Ca lactate. FIGS. 3A and 3B show the behavior of the 2 granulate just poured into HCl 0.1N (top view). On the left granulate with magnesium lactate and on the right granulate with calcium lactate. FIGS. 3A and 3B show the appearance of the 2 gelled granulates: granulate with Mg lactate and granulate with Ca lactate.

As can be seen from the photos, the melatonin granulate with calcium lactate, in contact with the HCl 0.1N solution, hydrates evenly and the powder granules gel smoothly and evenly, without forming aggregates. Granulate with magnesium lactate, on the other hand, hydrates with difficulty and longer times, and tends to form large lumpy aggregates, and, the part that hydrates, forms a gel with irregular characteristics and therefore unsuitable for the formation of a gel layer around the granular powder granule.

The gelling of the granulate and the proper dissolution of the granulate in the gummy matrix mass, during the gummy candy production process, are found to be crucial in achieving the controlled release of the active ingredient.

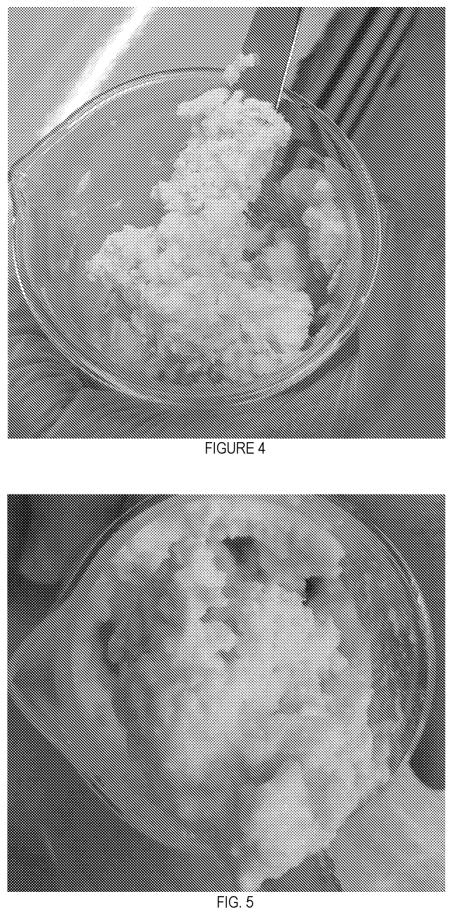

As can be seen from the photos (FIG. 4 and FIG. 5), simply dissolving the melatonin granuleate in water does not allow the granulate to gel to achieve controlled release.

The acidic pH of the solution used to deliver the melatonin granulate into the gummy candy matrix enhances the dissolution of the melatonin granules, promoting their gelling. Gelling is the precondition for achieving the modified release of the melatonin contained in the granulate.

### Example 5

Multilayer Gummy Composition with Melatonin

### Fast Layer

TABLE 6

| Trade name | mg/unit | | % |
|---|---|---|---|
| MELATONIN ID | 0.30 | mg | 0.81 |
| VITAMIN B6 HYDROCHLORIDE PYRID. | 2.04 | mg | 5.47 |
| VALERIAN DRY EXT. TIT 0.8%-1.0% AC. VALERENIC | 35.00 | mg | 93.72 |
| Total | 37.35 | mg | |

### Normal Layer

TABLE 7

| Trade name | mg/unit | | % |
|---|---|---|---|
| Lemon balm dryext. 10% Hydroxycinnamic tot. | 25.00 | mg | 41.67 |
| HAWTHORN DRY EXT. >1.8% VITEX. | 35.00 | mg | 58.33 |
| | 60.00 | mg | |

### Retard Layer

TABLE 8

| Trade name | mg/unit | | % |
|---|---|---|---|
| Melatonin Retard Granulate 10% | 7.01 | mg | 16.68 |
| PASSION FLOWER DRY EXT. TIT 3.5% VITEXIN | 35.00 | mg | 83.32 |
| Total | 42.01 | mg | |

Excipients of the gummy matrix: glucose syrup, sugar, water, food grade gelatin,
acidifier: citric acid, flavoring, coloring agents: E120, E131, E161b

### Example 6

Multilayer Gummy Composition with Vitamin C

### Retard Layer Composition

TABLE 9

| Trade name | mg/unit | | % |
|---|---|---|---|
| ascorbic acid retard granulate | 154.64 | mg | 100.00 |
| Total | 154.64 | mg | |

Normal “buffer” layer without vitamin C (in this formulation acts to reduce interactions between the 2 layers that contain vitamin C)

### Fast Layer

TABLE 10

| Trade name | mg/unit | | % |
|---|---|---|---|
| L-ASCORBIC ACID | 150.23 | mg | 100.00 |
| CRYSTALS (98% CRISTAL | | | |
| Total | 150.23 | mg | |

Excipients of the gummy matrix: glucose syrup, sugar, water, food grade gelatin,
acidifier: citric acid, flavoring, coloring agents: E120, E131, E161b

The preparation of ascorbic acid retard granulate involves granulation in a fluidized bed of a vitamin C grind with Compactcel F.

### Example 7

Multilayer Gummy Composition with Multivitamin

### Fast Layer

TABLE 11

| Trade name | mg/unit | | % |
|---|---|---|---|
| CALCIUM PANTOTHENATE | 2.04 | mg | 5.826 |
| VITAMIN PP NIACINAMIDE | 2.42 | mg | 6.936 |
| VITAMIN B1 NITRATE ROCOAT 33% OGM FREE | 1.24 | mg | 3.534 |
| VITAMIN B2 ROCOAT 33% RIBOFL. | 1.27 | mg | 3.642 |
| VITAMIN B6 HYDROCHLORIDE ROCOAT 33% | 1.55 | mg | 4.432 |
| FOLIC ACID | 0.07 | mg | 0.186 |
| VITAMIN B12 0.1% WS 429090 | 1.50 | mg | 4.292 |
| MAGNESIUM OXIDE CD | 24.87 | mg | 71.152 |
| | 34.95 | mg | |

### Normal Layer

TABLE 12

| Trade name | mg/unit | | % |
|---|---|---|---|
| ZINC BISGLYCINATE ID | 6.25 | mg | 34.04 |
| COPPER BISGLYCINATE ID | 0.94 | mg | 5.12 |
| CONTROLLED-RELEASE COATED | 8.98 | mg | 48.94 |
| FUMARATE IRON | | | |
| SODIUM SELENITE 45% | 0.07 | mg | 0.36 |
| CHROMIUM CHLORIDE 5% | 0.20 | mg | 1.09 |
| MANGANESE SULFATE H2O | 1.54 | mg | 8.38 |
| POTASSIUM IODIDE | 0.05 | mg | 0.27 |
| SODIUM MOLYBDATE 10% ID | 0.33 | mg | 1.81 |
| Total | 18.36 | mg | |

### Retard Layer

TABLE 13

| | | |
|---|---|---|
| Trade name | mg/unit | % |

| | | | |
|---|---|---|---|
| VITAMIN C GRANULATE | 74.23 | mg | 90.73 |
| VITAMIN A ACETATE 500 | 1.60 | mg | 1.96 |
| VITAMIN D3 100 CWS | 0.60 | mg | 0.73 |
| VITAMIN E ACETATE 50% TYPE SD | 5.38 | mg | 6.58 |
| BIOTIN (VITAMIN H) | 0.00 | mg | 0.00 |
| Total | 81.81 | mg | |

Excipients of the gummy matrix: glucose syrup, sugar, water, food gelatin, acidifier: citric acid, flavorings, coloring agents: E120, E131, E161b.

The retard ascorbic acid granulate is as shown in the previous example. Coated iron fumarate is hot prepared from iron (II) fumarate and glyceryl dibeenate (E471).

Method of Preparation of Coated Iron Fumarate with Controlled Release

The preparation of controlled-release coated iron fumarate involves the following operating procedure:
IRON FUMARATE: 60%
GLYCERYL DIBEENATE: 40%

In a suitable vessel, pour GLYCERYL DIBEENATE and thermostat at 60-65° C. until the product is completely melted;
Add FOLIC ACID and keep under stirring for 15 minutes until homogeneous dispersion;
Begin cooling and under slow stirring reach a product temperature of 40-45° C.;
Pour the product onto arella and let it cool down to room temperature or until solidified;
Manually crush the solid mixture obtained after complete cooling;
Grind the product with a mill equipped with 0.5 mm mesh and a coolant system by performing three steps:
1° step at speed 1
2° step at speed 2
3° step at speed 3
   Discharge the milled product

### Example 8

Multilayer Gummy Composition with Iron and Vitamin C

### Fast Layer

TABLE 14

| Trade name | mg/unit | | % |
|---|---|---|---|
| LIPOFER N.A. DISPERSIBLE (FE | 87.5, 00 | mg | 25.50 |
| PYROPHOSPHATE MICRONIZ) | | | |
| MALTODEXTRIN IT19 (GLUCIDEX) USP | 12.50 | mg | 12.50 |
| 0 | | mg | 0.00 |
| Total | 100.00 | mg | |

Normal “buffer” layer without vitamin C (in this formulation acts to reduce interactions between the 2 layers that contain Fe and Vitamin C.

### Retard Layer

TABLE 15

| Trade name | mg/unit | % |
|---|---|---|
| ascorbic acid retard granulate | 154.64 mg | 100.00 |
| Total | 154.64 mg | |

Excipients of the gummy matrix: glucose syrup, sugar, water, food gelatin, acidifier: citric acid, flavorings, coloring agents: E120, E131, E161b.

### Example 9

Multilayer Gummy Composition with Magnesium

### Fast Layer

TABLE 16

| Trade name | mg/unit | | % |
|---|---|---|---|
| MAGNESIUM OXIDE CD | 82.90 | mg | 82.90 |
| MALTODEXTRIN IT19 (GLUCIDEX) USP | 17.10 | mg | 17.10 |
| Total | 100.00 | mg | |

Normal “buffer” layer without magnesium (in this formulation acts to reduce interactions between the 2 layers that contain magnesium).

### Retard Layer

TABLE 17

| Trade name | mg/unit | | % |
|---|---|---|---|
| MAGNESIUM CITRATE ANHYDROUS | 157.83 | mg | 100.00 |
| Total | 157.83 | mg | |

Excipients of the gummy matrix: glucose syrup, sugar, water, food grade gelatin,

Acidifier: citric acid, flavoring, coloring agents: E120, E131, E161b

### Example 10

### Release Test of Vitamin C Gummy Composition (Example 6)

The composition obtained according to Example 6 is cut into pieces of about 2-3 mm and immersed in 900 mL HCl 0.1N, and is kept at 37° C., 100 rpm. The content of vitamin C
is analyzed over time up to 2 hours giving the following results.

TABLE 18

| | |
|---|---|
| Time | Vit C (%) |

| | | |
|---|---|---|
| 10 | min | 41% |
| 20 | min | 83% |
| 40 | min | 92% |
| 60 | min | 92% |
| 120 | min | 120% |

### Example 11

Release Test of Melatonin with Different Matrices.

A composition was prepared according to Example 5.

The same composition was repeated with the only difference being that 5% modified pea starch was added.

Dissolution times measured with Dissolution Test according to USP (500 mL HCl 0.1 N, 37° C., 100 rpm).

1. A chewable composition comprising a gummy matrix and at least one active ingredient into said gummy matrix, wherein said active ingredient comprises or alternatively consists of melatonin, sodium alginate, and calcium lactate.

2. The composition according to claim 1, wherein said matrix comprises a slow-release granulate comprising or alternatively consisting of melatonin, sodium alginate, and calcium lactate.

3. The composition according to claim 2, wherein said granulate comprises from 1% to 20% by weight of melatonin with respect to the total weight of the granulate, preferably from 1.5% to 15%, more preferably 2% to 10%; from 20% to 60% by weight of sodium alginate with respect to the total weight of the granulate, preferably from 40% to 55%, more preferably from 45% to 50%; and from 30 to 60% by weight of calcium lactate with respect to the total weight of the granulate, preferably from 40% to 55%, more preferably from 45% to 50%.

4. The composition according to claim 1, wherein said matrix has at least two layers, each of said layers containing one or more active ingredients, each layer being formulated with said at least one active ingredient comprising or alternatively consisting of melatonin, sodium alginate, and calcium lactate to provide different forms of controlled release of said at least one active ingredient.

5. The composition according to claim 1, wherein there are three superimposed layers in said matrix; preferably, at least one of the layers acts as a separator between the remaining layers; even more preferably, said separator layer is free of active ingredients.

6. The composition according to claim 5, wherein said matrix comprises a first immediate-release layer, a second slow-release layer, and a third intermediate-release layer.

7. The composition according to claim 1, wherein the same active ingredient is present in at least two layers in a differentiated release form.

8. The composition according to claim 1, wherein each layer contains different active ingredients.

9. The composition according to claim 1, wherein the gummy base formulation of at least two layers is different to provide a differentiated release of the active ingredient(s) present in said layer.

10. A process for the preparation of the composition according to claim 1, comprising the preparation of at least two layers of different formulations of one or more active ingredients in a single composition i.e., a gummy candy.

11. The process according to claim 10, wherein different layers have different formulation of said gummy matrix to provide a differentiated release of said one or more active ingredients.

12. The composition according to claim 1, wherein said composition is for use in a method of treatment of sleep disorders, preferably primary insomnia, for the reduction of the time required to fall asleep and for the maintenance of the state of falling asleep.

13. A method of treatment comprising administering the composition of claim 1 to a patient has a sleep disorder.

14. The method of claim 13, wherein the patient has insomnia.

15. The method according to claim 13, wherein said matrix comprises a slow-release granulate comprising or alternatively consisting of melatonin, sodium alginate, and calcium lactate.

16. The method according to claim 15, wherein said granulate comprises from 1% to 20% by weight of melatonin with respect to the total weight of the granulate, preferably from 1.5% to 15%, more preferably 2% to 10%; from 20% to 60% by weight of sodium alginate with respect to the total weight of the granulate, preferably from 40% to 55%, more preferably from 45% to 50%; and from 30 to 60% by weight of calcium lactate with respect to the total weight of the granulate, preferably from 40% to 55%, more preferably from 45% to 50%.

17. The method according to claim 13, wherein said matrix has at least two layers, each of said layers containing one or more active ingredients, each layer being formulated with said at least one active ingredient comprising or alternatively consisting of melatonin, sodium alginate, and calcium lactate to provide different forms of controlled release of said at least one active ingredient.

18. The method according to claim 13, wherein there are three superimposed layers in said matrix; preferably, at least one of the layers acts as a separator between the remaining layers; even more preferably, said separator layer is free of active ingredients.

19. The method according to claim 18, wherein said matrix comprises a first immediate-release layer, a second slow-release layer, and a third intermediate-release layer.

20. The method according to claim 13, wherein the same active ingredient is present in at least two layers in a differentiated release form.
